(19) United States
(12) Patent Application Publication
(10) Pub. No.: US 2010/0216690 A1
(43) Pub. Date: Aug. 26, 2010

(54) Pegylated Single-Chain Insulin

(75) Inventors: Peter Madsen, Bagsvaerd (DK); Thomas Borglum Kjeldsen, Virum (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Baagsvaerd (DK)

(21) Appl. No.: 11/908,646

(22) PCT Filed: Mar. 17, 2006
(86) PCT No.: PCT/EP06/60816

§ 371 Date: Oct. 15, 2007

Related U.S. Application Data
(60) Provisional application No. 60/668,817, filed on Apr. 6, 2005.

(30) Foreign Application Priority Data
Mar. 18, 2005 (DK) PA 2005 00400

(51) Int. Cl. A61K 38/28; C07K 14/62; A61P 3/10

(52) U.S. Cl. 514/3; 530/303

(57) ABSTRACT

The present invention is related to a single-chain insulin comprising the B- and the A-chain of human insulin or analogues thereof connected by a connecting peptide having from 3-35 amino acid residues, wherein the single-chain insulin comprises at least one PEG group attached to at least one lysine residue in the single-chain insulin molecule and/or to the B1 N terminal amino acid residue. The PEGylated single-chain insulins may comprise up to 4 PEG groups which may be the same or different.

### FIELD OF THE INVENTION

The present invention is related to PEGylated single-chain insulins which have insulin activity and can be used for the treatment of diabetes. The PEGylated single-chain insulins have higher bioavailability and a longer time-action profile than regular insulin and are in particular suited for pulmonal administration. They will also have a high physical stability and a low tendency to fibrillation and will be soluble at neutral pH. The present invention is also related to pharmaceutical compositions containing the pegylated single-chain insulins.

### BACKGROUND OF THE INVENTION

Insulin is a polypeptide hormone secreted by β-cells of the pancreas and consists of two polypeptide chains, A and B, which are linked by two inter-chain disulphide bridges. Furthermore, the A-chain features one intra-chain disulphide bridge.

The hormone is synthesized as a single-chain precursor proinsulin (preproinsulin) consisting of a prepeptide of 24 amino acid followed by proinsulin containing 86 amino acids in the configuration: prepeptide-B-Arg Arg-C-Lys Arg-A, in which C is a connecting peptide of 31 amino acids. Arg-Arg and Lys-Arg are cleavage sites for cleavage of the connecting peptide from the A and B chains to form the two-chain insulin molecule. Insulin is essential in maintaining normal metabolic regulation.

The two chain structure of insulin allows insulin to undertake multiple conformations, and several findings have indicated that insulin has the propensity to considerable conformational change and that restrictions in the potential for such change considerably decrease the affinity of the insulin receptor for ligands. Proinsulin has a 100 fold lower affinity for the insulin receptor than native insulin. Blocking of the amino acid residue A1 in insulin also results in poor receptor binding, consistent with the dogma that a free N-terminal of the A-chain and free C-terminal of the B-chain of insulin are important for binding to the insulin receptor.

The inherited physical and chemical stability of the insulin molecule is a basic condition for insulin therapy of diabetes mellitus. These basic properties are fundamental for insulin formulation and for applicable insulin administration methods, as well as for shelf-life and storage conditions of pharmaceutical preparations. Use of solutions in administration of insulin exposes the molecule to a combination of factors, e.g. elevated temperature, variable air-liquid-solid interphases as well as shear forces, which may result in irreversible conformation changes e.g. fibrillation.

Unfortunately, many diabetics are unwilling to undertake intensive therapy due to the discomfort associated with the many injections required to maintain close control of glucose levels. This type of therapy can be both psychologically and physically painful. Upon oral administration, insulin is rapidly degraded in the gastro intestinal tract and is not absorbed into the blood stream. Therefore, many investigators have studied alternate routes for administering insulin, such as oral, rectal, transdermal, and nasal routes. Thus far, however, these routes of administration have not resulted in effective insulin absorption.

Efficient pulmonary delivery of a protein is dependent on the ability to deliver the protein to the deep lung alveolar epithelium. Proteins that are deposited in the upper airway epithelium are not absorbed to a significant extent. This is due to the overlying mucus which is approximately 30-40 μm thick and acts as a barrier to absorption. In addition, proteins deposited on this epithelium are cleared by mucociliary transport up the airways and then eliminated via the gastrointestinal tract. This mechanism also contributes substantially to the low absorption of some protein particles. The extent to which proteins are not absorbed and instead eliminated by these routes depends on their solubility, their size, as well as other less understood characteristics.

It is however well recognised that the properties of peptides can be enhanced by grafting organic chain-like molecules onto them. Such grafting can improve pharmaceutical properties such as half life in serum, stability against proteolytical degradation, and reduced immunogenicity.

The organic chain-like molecules often used to enhance properties are polyethylene glycol-based chains, i.e., chains that are based on the repeating unit —CH₂CH₂O—. Hereinafter, the abbreviation “PEG” is used for polyethyleneglycol.

Classical PEG technology takes advantage of providing polypeptides with increased size (Stoke radius) by attaching a soluble organic molecule to the polypeptide (Kochendoerfer, G., et al., Science (299) 884-, 2003). This technology leads to reduced clearance in man and animals of a hormone polypeptide compared to the native polypeptide. However this technique is often hampered by reduced potency of the hormone polypeptides subjected to this technique (Hinds, K., et al., Bioconjugate Chem. (11), 195-201, 2000).

Insulin compositions for pulmonary administration comprising a conjugate of two-chain insulin covalently coupled to one or more molecules of non-naturally hydrophilic polymers including polyalkylene glycols and methods for their preparation are disclosed in WO 02/094200 and WO 03/022996.

However, there is still a need for insulins having a more prolonged profile of action than the insulin derivatives known up till now and which at the same time are soluble at physiological pH values and have a potency which is comparable to that of human insulin. Furthermore, there is need for further insulin formulations which are well suited for pulmonary application.

### SUMMARY OF THE INVENTION

In one aspect the present invention is related to a single-chain insulin comprising the B- and the A-chain of human insulin or analogues thereof connected by a connecting peptide having from 3-35 amino acid residues, wherein the single-chain insulin comprises at least one PEG group attached to at least one lysine residue in the single-chain insulin molecule and/or to the B-chain N-terminal amino acid residue.

The PEG group will be attached to a lysine residue in the parent single-chain insulin via a suitable linker group. The linker is typically a derivative of a carboxylic acid, where the carboxylic acid functionality is used for attachment to insulin via an amide bond. The linker may be an acetic acid with the linking motif: —CH₂CO—, a propionic acid with the linking motif: —CH₂CH₂CO— or —CHCH₃CO—, or a butyric acid with the linking motif: CH₂CH₂CH₂CO— or —CH₂CHCH₃CO—. The linker may also be a —CO— group.

In one embodiment the PEG group is attached to the naturally occurring lysine residue in the parent insulin molecule, the B29 lysine residue. Alternatively, the PEG group is attached to a lysine residue substituted for a natural amino acid residue in selected positions in the B- or A-chain of the parent insulin molecule. The PEG group may also be attached to a lysine residue in the connecting peptide. Finally, the PEG group may be attached to the N-terminal amino acid group of the B chain, for example the B1 position. The PEG group may in this case be attached to the free amino group in the natural Phe residue in position B1 or the natural Phe residue may by substituted with another naturally occurring amino acid or may be deleted.

If the single-chain insulin comprises more than one PEG group the PEG groups may be attached to any combination of the selected amino acids in the parent insulin molecule.

In one embodiment of the invention the single-chain insulin comprises at least one PEG group which is attached to a lysine residue in one or more of positions B1; B2; B3; B4; B10; B20; B21; B22; B27; B28; B29; B30; A8; A9; A10; A14; A15; A18; A21; A22; A23 in the parent single-chain insulin molecule and/or in the connecting peptide. The PEGylated, single-chain insulins according to the invention may comprise up to 4 PEG groups which may be the same or different. Thus the single-chain can have 1, 2, 3, or 4 PEG groups attached to the molecule.

In one embodiment the single-chain insulin has only one PEG group attached to the insulin molecule.

In one embodiment of the invention the PEG group is attached to a lysine residue in position B1 in the parent single-chain insulin molecule.

In one embodiment of the invention the PEG group is attached to a lysine residue in position B2 in the parent single-chain insulin molecule.

In one embodiment of the invention the PEG group is attached to a lysine residue in position B3 in the parent single-chain insulin molecule.

In one embodiment of the invention the PEG group is attached to a lysine residue in position B4 in the parent single-chain insulin molecule.

In one embodiment of the invention the PEG group is attached to a lysine residue in position B10 in the parent single-chain insulin molecule.

In one embodiment of the invention the PEG group is attached to a lysine residue in position B20 in the parent single-chain insulin molecule.

In one embodiment of the invention the PEG group is attached to a lysine residue in position B21 in the parent single-chain insulin molecule.

In one embodiment of the invention the PEG group is attached to a lysine residue in position B22 in the parent single-chain insulin molecule.

In one embodiment of the invention the PEG group is attached to a lysine residue in position B27 in the parent single-chain insulin molecule.

In one embodiment of the invention the PEG group is attached to a lysine residue in position B28 in the parent single-chain insulin molecule.

In one embodiment of the invention the PEG group is attached to a lysine residue in position B29 in the parent single-chain insulin molecule.

In one embodiment of the invention the PEG group is attached to a lysine residue in position B30 in the parent single-chain insulin molecule.

In one embodiment of the invention the PEG group is attached to a lysine residue in position A8 in the parent single-chain insulin molecule.

In one embodiment of the invention the PEG group is attached to a lysine residue in position A9 in the parent single-chain insulin molecule.

In one embodiment of the invention the PEG group is attached to a lysine residue in position A10 in the parent single-chain insulin molecule.

In one embodiment of the invention the PEG group is attached to a lysine residue in position A14 in the parent single-chain insulin molecule.

In one embodiment of the invention the PEG group is attached to a lysine residue in position A15 in the parent single-chain insulin molecule.

In one embodiment of the invention the PEG group is attached to a lysine residue in position A18 in the parent single-chain insulin molecule.

In one embodiment of the invention the PEG group is attached to a lysine residue in position A21 in the parent single-chain insulin molecule.

In one embodiment of the invention the PEG group is attached to a lysine residue in position A22 in the parent single-chain insulin molecule.

In one embodiment of the invention the PEG group is attached to a lysine residue in position A23 in the parent single-chain insulin molecule.

In one embodiment of the invention the PEG group is attached to a lysine residue in position B20, B21 or B22.

In one embodiment of the invention the PEG group is attached to a lysine residue in position B27; B28; B29; or B30.

In one embodiment of the invention the PEG group is attached to a lysine residue in position B1; B2; B3; B4.

In one embodiment of the invention the PEG group is attached to a lysine residue in position B10.

In one embodiment of the invention the PEG group is attached to a lysine residue in position A8, A9 or A10.

In one embodiment of the invention the PEG group is attached to a lysine residue in position A14, A15 or A18.

In one embodiment of the invention the PEG group is attached to a lysine residue in position A21, A22 or A23.

In one embodiment of the invention the PEG group is attached to a lysine residue in position A22 or A23.

In another embodiment of the invention the PEG group is attached to a lysine residue in the connecting peptide.

If PEGylation of the natural lysine group in position B29 in the insulin B-chain is unwanted this amino acid residue may be replaced by another amino acid residue. Suitable replacement amino acid residues are Ala, Arg, Gln and His. Alternatively the lysine amino acid residue in position B29 may be blocked by well known technology before PEGylation of the lysine residue in the desired position in the insulin molecule followed by deblocking after PEGylation.

In one embodiment the PEGylated single-chain insulin has an Ala, Arg, Gln or His amino acid residue substituted for the natural Lys residue in position B29 in the B chain.

In another embodiment the PEGylated single-chain insulin has an Ala amino acid residue substituted for the natural Lys residue in position B29 in the B chain.

In another embodiment the PEGylated single-chain insulin has an Arg amino acid residue substituted for the natural Lys residue in position B29 in the B chain.

In another embodiment the PEGylated single-chain insulin has a Gln amino acid residue substituted for the natural Lys residue in position B29 in the B chain.

In another embodiment the PEGylated single-chain insulin has a His amino acid residue substituted for the natural Lys residue in position B29 in the B chain.

The parent single-chain insulin molecule may have a limited number of the naturally occurring amino acid residues substituted with other amino acid residues as explained in the detailed part of the specification.

Thus in one embodiment of the invention the single-chain insulin has the natural amino acid residue in position A18 substituted with a Gln residue.

In another embodiment of the invention the single-chain insulin has the natural amino acid residue in position A21 substituted with a Gly residue.

In a further embodiment of the invention the single-chain insulin has the natural amino acid residue in position B30 substituted with another amino acid residue or B30 is deleted.

The length of the connecting peptide may vary from 3 amino acid residues and up to a length corresponding to the length of the natural C-peptide in human insulin. The connecting peptide in the PEGylated, single-chain insulins according to the present invention is however normally shorter than the human C-peptide and will typically have a length from 5-20, from 5-18, from 5-16, from 5-15 or from 5-11 amino acid residues.

Alternatively, the connecting peptide has from 3 to about 35, from 3 to about 30, from 4 to about 35, from 4 to about 30, from 5 to about 35, from 5 to about 30, from 6 to about 35 or from 6 to about 30, from 3 to about 25, from 3 to about 20, from 4 to about 25, from 4 to about 20, from 5 to about 25, from 5 to about 20, from 6 to about 25 or from 6 to about 20, from 3 to about 15, from 3 to about 10, from 4 to about 15, from 4 to about 10, from 5 to about 10, from 6 to about 15 or from 6 to about 10.

In one embodiment of the present invention the connecting peptide has from 6-10, 6-9, 6-8, 6-7, 7-8, 7-9, or 7-10 amino acid residues in the peptide chain.

Examples of connection peptides which may be suitable for the present PEGylated single-chain insulins are disclosed in WO 2005/054291.

In a further embodiment the connecting peptide is selected from the group consisting of TGLGSGQ (SEQ ID NO:1); VGLSSGQ (SEQ ID NO:2); VGLSSGK (SEQ ID NO:3); TGLGSGR (SEQ ID NO:4); TGLGKGQ (SEQ ID NO:5); KGLSSGQ (SEQ ID NO:6); VKLSSGQ (SEQ ID NO:7); VGLKSGQ (SEQ NO:8); TGLGKGQ (SEQ ID NO:9) and VGLSKGQ (SEQ ID NO:10).

The PEG group may vary in size within a large range as it well known within the art. However, too large PEG groups may interfere in a negative way with the biological activity of the PEGylated single-chain insulin molecule.

Non limiting examples of PEG groups are such comprising a number of (OCH₂CH₂) subunits from 800 to about 1000; from 850 to about 950; from 600 to about 700; from about 400 to about 500; from about 180 to about 300; from about 100 to about 150; from about 35 to about 55; from about 42 to about 62; or from about 12 to about 25 subunits.

In one embodiment the PEG groups have the formula CH₃O(CH₂CH₂O)ₙCH₂CH₂—O—, where n is an integer from 2 to about 600.

In another embodiment n may be from about 400 to about 500.

The parent insulin molecule may be characterized by the formula

(SEQ ID NO: 11)

B1-B2-B3-B4-His-Leu-Cys-Gly-Ser-B10-Leu-Val-Glu-
Ala-Leu-Tyr-Leu-Val-Cys-B20-B21-B22-Gly-Phe-Phe-
Tyr-B27-B28-B29-B30-Cx-Gly-Ile-Val-Glu-Gln-Cys-
Cys-A8-A9-Ile-Cys-Ser-Leu-Tyr-A15-Leu-Glu-A18-
Tyr-Cys-A21-A22-A23

wherein B1 is Phe, Lys, Asp or is deleted; B2 is Val or Lys; B3 is Asn, Ser; Thr, Lys, Gln, Glu or Asp; B4 is Gln or Lys; B10 is His, Gln, or Lys, B20 is Gly or Lys, B21 is Glu or Lys, B22 is Arg or Lys, B27 is Thr, Arg, Glu or Lys, B28 is Pro, Asp, Ile or Lys, B29 is Lys, Arg, Ala, Asp, Phe, Tyr, His, Gln or Pro, B30 is Thr, Lys or a peptide bond, Cx is a peptide chain (connecting peptide) of 3-35 amino acid residues, A8 is Thr or Lys, A9 is Ser or Lys, A15 is Gln or Lys, A18 is Asn, Gln or Lys, A21 is Asn, Ala, Gln, Glu, Gly, His, Ile, Leu, Met, Ser, Thr, Trp, Tyr, Lys or Val; A22 is any amino acid residue including Lys or is absent and A23 is Lys or is absent with the proviso that the maximum number of lysine residue in the parent single-chain insulin is 4.

In one embodiment the maximum number of lysine residues is three.

In another embodiment the maximum number of lysine residues is two and in a further embodiment there is only one lysine residue in the parent single-chain insulin.

In one embodiment Cx is a peptide sequence with the following formula Xₐ-X_{b}-X_{c}-X_{d}-Xₑ-X_{f}-X_{g} (SEQ ID NO:12) wherein

Xₐ is selected from the group consisting of L, R, T, A, H, Q, G, S and V;

X_{b} is selected from the group consisting of W, G, S, A, H, R, and T;

X_{c} is selected from the group consisting of L, Y, M, H, R, T, Q, K, V, S, A, G and P;

X_{d} is selected from the group consisting of R, A, Y, M, S, N, H, and G;

Xₑ is selected from the group consisting of S, R, A, T, K P, N M, H, Q, V, and G;

X_{f} is selected from the group consisting of G and A; and

X_{g} is selected from the group consisting of K, R, P, H, F, T, I, Q, W, and A

In a further embodiment

Xₐ is selected from the group consisting of L, R, T, A, H and V;

X_{b} is selected from the group consisting of W, G, S, A, H, R, and T;

X_{c} is selected from the group consisting of L, Y, M, H, R, T, Q, K, V, S, A, G and P;

X_{d} is selected from the group consisting of R, A, Y, M, S, N, H, and G;

Xₑ is selected from the group consisting of S, R, A, T, K P, and N;

X_{f} is G; and

X_{g} is selected from the group consisting of K, R, Q and P;

In a further embodiment

Xₐ is selected from the group consisting of T, A V, K;

X_{b} is G;

X_{c} is selected from the group consisting of L, Y, M, H, R K, W;

X_{d} is G;

Xₑ is selected from the group consisting of S, K;

X_{f} is G, and

X_{g} is selected from the group consisting of K, R, Q.

In a still further embodiment Cx has the sequence X₉-G-X₁₀-G-X₁₁-G-X₁₂ (SEQ ID NO:13)

wherein

X₉ selected from the group consisting of Val, Leu, Arg, Thr, Ala, His, Gln, Gly or Ser,

X₁₀ is selected from the group consisting of Leu, Tyr, Met, His, Arg, Thr, Gln, Lys, Val, Ser, Ala, Gly, Pro,

X₁₁ is selected from the group consisting of Ser, Arg, Ala, Thr, Lys, Pro, Asn, Met, His, Gln, Val, Gly, and

X₁₂ is Lys or Arg.

In a still further embodiment X₉ selected from the group consisting of Val, Leu, Arg, Thr, Ala, and His,

X₁₀ is selected from the group consisting of Leu, Tyr, Met, and His,

X₁₁ is selected from the group consisting of Ser, Arg, Ala, Thr, Lys, Pro and Asn and

X₁₂ is Lys or Arg.

In another embodiment the single-chain insulin is a desB1, desB25, desB27, desB28 or desB29 insulin analogue.

In another embodiment the connecting peptide has a GR or GQ di-peptide sequence attached to the A1 amino acid residue.

In still a further aspect the present invention is related to pharmaceutical preparations comprising the PEGylated, single-chain insulin of the invention and suitable adjuvants and additives such as one or more agents suitable for stabilization, preservation or isotoni, for example, zinc ions, phenol, cresol, a parabene, sodium chloride, glycerol or mannitol. The zinc content of the present formulations may be between 0 and about 6 zinc atoms per insulin hexamer. The pH of the pharmaceutical preparation may be between about 4 and about 8.5, between about 4 and about 5 or between about 6.5 and about 7.5.

In a further embodiment the present invention is related to the use of the PEGylated, single-chain insulin as a pharmaceutical for the reducing of blood glucose levels in mammalians, in particularly for the treatment of diabetes.

In a further aspect the present invention is related to the use of the PEGylated, single-chain insulin for the preparation of a pharmaceutical preparation for the reducing of blood glucose level in mammalians, in particularly for the treatment of diabetes.

In a further embodiment the present invention is related to a method of reducing the blood glucose level in mammalians by administrating a therapeutically active dose of a PEGylated, single-chain insulin according to the invention to a patient in need of such treatment.

In a further aspect of the present invention the PEGylated, single-chain insulins are administered in combination with one or more further active substances in any suitable ratios. Such further active agents may be selected from human insulin, fast acting insulin analogues, antidiabetic agents, antihyperlipidemic agents, antiobesity agents, antihypertensive agents and agents for the treatment of complications resulting from or associated with diabetes.

In one embodiment the two active components are administered as a mixed pharmaceutical preparation. In another embodiment the two components are administered separately either simultaneously or sequentially.

In one embodiment the PEGylated, single-chain insulins of the invention may be administered together with fast acting human insulin or human insulin analogues. Such fast acting insulin analogue may be such wherein the amino acid residue in position B28 is Asp, Lys, Leu, Val, or Ala and the amino acid residue in position B29 is Lys or Pro, des(B28-B30), des(B27) or des(B30) human insulin, and an analogue wherein the amino acid residue in position B3 is Lys and the amino acid residue in position B29 is Glu or Asp. The PEGylated, single-chain insulin according to the invention and the rapid acting human insulin or human insulin analogue can be mixed in a ratio from about 90/10%; about 70/30% or about 50/50%.

Antidiabetic agents will include insulin, GLP-1(1-37) (glucagon like peptide-1) described in WO 98/08871, WO 99/43706, U.S. Pat. No. 5,424,286 and WO 00/09666, GLP-2, exendin-4(1-39), insulinotropic fragments thereof, insulinotropic analogues thereof and insulinotropic derivatives thereof. Insulinotropic fragments of GLP-1(1-37) are insulinotropic peptides for which the entire sequence can be found in the sequence of GLP-1(1-37) and where at least one terminal amino acid has been deleted.

The PEGylated single-chain insulins according to the present invention may also be used on combination treatment together with an oral antidiabetic such as a thiazolidindione, metformin and other type 2 diabetic pharmaceutical preparation for oral treatment.

Furthermore, the PEGylated, single-chain insulin according to the invention may be administered in combination with one or more antiobesity agents or appetite regulating agents.

In one embodiment the invention is related to a pulmonal pharmaceutical preparation comprising the PEGgylated single-chain insulin of the invention and suitable adjuvants and additives such as one or more agents suitable for stabilization, preservation or isotoni, for example, zinc ions, phenol, cresol, a parabene, sodium chloride, glycerol, propyleneglycol or mannitol.

It should be understood that any suitable combination of the PEGylated, single-chain insulins with diet and/or exercise, one or more of the above-mentioned compounds and optionally one or more other active substances are considered to be within the scope of the present invention.

### DETAILED DESCRIPTION OF THE INVENTION

The stability and solubility properties of insulin are important underlying aspects for current insulin therapy. The present invention is addressed to these issues by providing stable, PEGylated single-chain insulin analogues wherein the introduction of a connecting peptide between the B- and A-chain decreases molecular flexibility and concomitantly reduce the fibrillation propensity and limit or modify the pH precipitation zone.

The PEGylated single-chain insulins according to the invention are in particularly intended for pulmonal administration due to their relatively high bioavailability compared to eg. human insulin. Furthermore, the PEGylated single-chain insulins will have a protracted insulin activity.

The PEGgylated single-chain insulins according to the invention for administration to the lung may have PEG groups with a molecular weight varying within a rather broad range. The molecular weight ranges will typically be from about 4500 to about 5500 dalton, from about 3500 to about 4500 dalton, from about 2500 to about 3500 dalton, from about 1500 to about 2500 dalton, from about 750 to about 1500 dalton and from about 500 to about 1000 daltons.

Non limiting examples of average molecular weights of the PEG moieties are 500, 600, 700, 750, 800, 900, 1000, 1500, 2000, 2300, 2500, 3000, 4000 and 5000 dalton.

Because virtually all PEG polymers are mixtures of many large molecules, one must resort to averages to describe molecular weight. Among many possible ways of reporting averages, three are commonly used: the number average, weight average, and z-average molecular weights. The weight average is probably the most useful of the three, because it fairly accounts for the contributions of different sized chains to the overall behaviour of the polymer, and correlates best with most of the physical properties of interest.

$$Number average MW ( {\overline{M}}_{n} ) . \frac{Σ ( {M}_{i} {N}_{i} )}{\sum ( {N}_{i} )}$$

$$Weight average MW ( {\overline{M}}_{w} ) . \frac{Σ ( {M}_{i}^{2} {N}_{i} )}{Σ ( {M}_{i} {N}_{i} )}$$

$$Z average MW ( {\overline{M}}_{z} ) . \frac{Σ ( {M}_{i}^{3} {N}_{i} )}{Σ ( {M}_{i}^{2} {N}_{i} )}$$

where Nᵢ is the mole-fraction (or the number-fraction) of molecules with molecular weight Mᵢ in the polymer mixture. The ratio of M_{w} to Mₙ is known as the polydispersity index (PDI), and provides a rough indication of the breadth of the distribution. The PDI approaches 1.0 (the lower limit) for special polymers with very narrow MW distributions.

While lower molecular weight PEG groups may be preferred for increasing bioavailability, high molecular weight PEG chains, e.g., having an average molecular weight of 4000-6000 daltons or greater, although generally found to decrease the bioactivity of the insulin molecule, may be preferred for increasing half-life, particularly in the case of injectable formulations.

The PEG groups of the present invention will typically comprise a number of (OCH₂CH₂) subunits e.g. from 2 to about 600 subunits, from about 4 to about 200 subunits, from about 4 to about 170 subunits, from about 4 to about 140 subunits, from about 4 to about 100 subunits, from about 10 to about 100 subunits, from about 4 to about 70 subunits, from about 4 to about 45 subunits, and from about 4 to about 25 subunits.

Well suited PEG groups are such wherein the number of subunits are selected from the group consisting of from about 800 to about 1000; from about 850 to about 950; from about 600 to about 700; from about 400 to about 500; from about 180 to about 300; from about 100 to about 150; from about 35 to about 55; from about 42 to about 62; or from about 12 to about 25 subunits.

The PEG groups of the invention will for a given molecular weight typically consist of a range of ethyleneglycol (or ethyleneoxide) monomers. For example, a PEG group of molecular weight 2000 dalton will typically consist of 43±10 monomers, the average being around 43-44 monomers.

The PEG-moieties (including mPEG-) are attached to the parent single chain insulin molecule via a suitable linker. This linker is typically a derivative of a carboxylic acid, where the carboxylic acid functionality is used for attachment to insulin via an amide bond. The linker, for example, is acetic acid (linking motif: —CH₂CO—), propionic acid (linking motif: —CH₂CH₂CO— or —CHCH₃CO—), butyric acid (linking motif: —CH₂CH₂CH₂CO— or —CH₂CHCH₃CO—). The linker can also be —CO—.

The insulin molecule which is PEGylated according to the present invention is a single-chain insulin molecule wherein the A and B chain of insulin is connected by a connecting peptide of up to 35 amino acid residues in length. However, the connecting peptide will typically be shorter than the natural connecting peptide and may be as short as 3 amino acid residues long.

The PEGgylated single-chain insulins according to the present invention may be mono-substituted having only one PEG group attached to a lysine amino acid residue in the parent insulin molecule. Alternatively the PEGylated single-chain insulins according to the present invention may comprise two, three- or four PEG groups. If the single-chain insulin comprises more than one PEG group it will typically have same PEG moiety attached to each lysine group. However, the individual PEG groups may also vary from each other in size and length.

The only natural lysine residue in the human insulin A and B chain is the lysine residue in position B29. If a PEG group is to be attached at another position in the parent single-chain insulin molecule it is necessary to substitute a lysine residue for the natural residue at the position in question. This is done by well known technology as it appears from the following. Suitable amino acid substitutes are Ale, Arg, Gln and His.

The parent single-chain insulins are named according to the following rule: The sequence starts with the B-chain, continues with the connecting peptide and ends with the A-chain. The amino acid residues are named after their respective counterparts in human insulin and mutations and PEGylations are explicitly described whereas unaltered amino acid residues in the A- and B-chains are not mentioned. For example, an single-chain insulin having the following mutations as compared to human insulin: A21G, A18Q, B3Q, B29R, desB30 and the connecting peptide TGLGKGQ (SEQ ID NO:5) connecting the C-terminal B-chain and the N-terminal A-chain and being PEGylated in the lysine residue in the connecting peptide with mPEG-propionic acid, 2 kDa eg. using mPEG-SPA is named B(1-29)-B3Q-B29R-TGLGK(N^{ε}-(3-(mPEG2000-yl)-propionyl)GQ-A(1-21)-A18Q-A21G human insulin.

Non limiting examples of parent single-chain insulin molecules are such wherein

the amino acid residue in position B27 is K, the amino acid residue in position B29 is A and the amino acid residue in position A18 is Q;

the amino acid residue in position B29 is K and the amino acid residue in position A18 is Q;

the amino acid residue in position B18 is K, the amino acid residue in position B29 is A and the amino acid residue in position A18 is Q;

the amino acid residue in position B28 is K, the amino acid residue in position B29 is A and the amino acid residue in position A18 is Q;

the amino acid residue in position B3 is K, the amino acid residue in position B29 is A and the amino acid residue in position A18 is Q;

the amino acid residue in position B10 is K, the amino acid residue in position B29 is A and the amino acid residue in position A18 is Q;

the amino acid residue in position B22 is K, the amino acid residue in position B29 is A and the amino acid residue in position A18 is Q;

the amino acid residue in position A8 is K, the amino acid residue in position B29 is A and the amino acid residue in position A18 is Q;

the amino acid residue in position A9 is K, the amino acid residue in position B29 is A and the amino acid residue in position A18 is Q;

the amino acid residue in position A22 is K, the amino acid residue in position B29 is A and the amino acid residue in position A18 is Q;

the amino acid residue in position A23 is K, the amino acid residue in position B29 is A and the amino acid residue in position A18 is Q;

the amino acid residue in position A15 is K, the amino acid residue in position B29 is A and the amino acid residue in position A18 is Q;

the amino acid residue in position B29 is A and the amino acid residue in position A18 is K;

the amino acid residue in position B27 is K and the amino acid residue in position B29 is A;

the amino acid residue in position B29 is K;

the amino acid residue in position B18 is K and the amino acid residue in position B29 is A;

the amino acid residue in position B28 is K and the amino acid residue in position B29 is A;

the amino acid residue in position B3 is K and the amino acid residue in position B29 is A;

the amino acid residue in position B10 is K and the amino acid residue in position B29 is A;

the amino acid residue in position B22 is K and the amino acid residue in position B29 is A;

the amino acid residue in position A8 is K and the amino acid residue in position B29 is A;

the amino acid residue in position A9 is K and the amino acid residue in position B29 is A;

the amino acid residue in position A22 is K and the amino acid residue in position B29 is A;

the amino acid residue in position A23 is K and the amino acid residue in position B29 is A;

the amino acid residue in position A15 is K and the amino acid residue in position B29 is A; or

the amino acid residue in position B29 is A and the amino acid residue in position A18 is K.

Examples of parent, single-chain insulins of the invention include:

B(1-29)-B29A-VGLSSGQ-A(1-21)-A18Q Human insulin;
B(1-29)-B3K-B29A-VGLSSGQ-A(1-21)-A18Q Human insulin;
B(1-29)-B22K-B29A-VGLSSGQ-A(1-21)-A18Q Human insulin;
B(1-29)-B27K-B29A-VGLSSGQ-A(1-21)-A18Q Human insulin;
B(1-29)-B28K-B29A-VGLSSGQ-A(1-21)-A18Q Human insulin;
B(1-29)-VGLSSGQ-A(1-21)-A18Q Human insulin;
B(1-29)-B29A-KGLSSGQ-A(1-21)-A18Q Human insulin;
B(1-29)-B29A-VKLSSGQ-A(1-21)-A18Q Human insulin;
B(1-29)-B29A-VGLKSGQ-A(1-21)-A18Q-A21G Human insulin;
B(1-29)-B29A-TGLGKGQ-A(1-21)-A18Q Human insulin;
B(1-29)-B29A-VGLSKGQ-A(1-21)-A18Q-A21G Human insulin;
B(1-29)-B29A-VGLSSGK-A(1-21)-A18Q-A21G Human insulin;
B(1-29)-B29A-VGLSSGQ-A(1-21)-A8K-A18Q Human insulin;
B(1-29)-B29A-VGLSSGQ-A(1-21)-A15K-A18Q Human insulin;
B(1-29)-B29A-VGLSSGQ-A(1-21)-A18K Human insulin;
B(1-29)-B29A-VGLSSGQ-A(1-21)-A18Q-A22K Human insulin;
B(1-29)-B29A-TGLGSGR-A(1-21)-A18Q-A21G Human insulin;
B(1-29)-B290-TGLGSGR-A(1-21)-A18Q-A21G Human insulin;
B(1-29)-B29H-TGLGSGR-A(1-21)-A18Q-A21G Human insulin;
B(1-29)-B3K-B29A-TGLGSGR-A(1-21)-A18Q-A21G Human insulin;
B(1-29)-B3K-B290-TGLGSGR-A(1-21)-A18Q-A21G Human insulin;
B(1-29)-B3K-B29H-TGLGSGR-A(1-21)-A18Q-A21G Human insulin;
B(1-29)-B20K-B290-TGLGSGR-A(1-21)-A18Q-A21G Human insulin;
B(1-29)-B21K-B290-TGLGSGR-A(1-21)-A18Q-A21G Human insulin;
B(1-29)-B22K-B29H-TGLGSGR-A(1-21)-A18Q-A21G Human insulin;
B(1-29)-B22K-B29H-TGLGSGR-A(1-21)-A18Q Human insulin;
B(1-29)-B22K-B290-TGLGSGR-A(1-21)-A18Q-A21G Human insulin;
B(1-29)-B22K-B290-TGLGSGR-A(1-21)-A18Q Human insulin;
B(1-29)-B22K-B29A-TGLGSGR-A(1-21)-A18Q-A21G Human insulin;
B(1-29)-B22K-B29A-TGLGSGR-A(1-21)-A18Q Human insulin;
B(1-29)-B22K-B29S-TGLGSGR-A(1-21)-A18Q-A21G Human insulin;
B(1-29)-B22K-B29S-TGLGSGR-A(1-21)-A18Q Human insulin; B(1-29)-B29A-TGLGSGR-A(1-21)-A8K-A18Q-A21G Human insulin;
B(1-29)-B290-TGLGSGR-A(1-21)-A8K-A18Q-A21G Human insulin;
B(1-29)-B29H-TGLGSGR-A(1-21)-A8K-A18Q-A21G Human insulin;
B(1-29)-B29A-TGLGSGR-A(1-21)-A18K-A21G Human insulin;
B(1-29)-B290-TGLGSGR-A(1-21)-A18K-A21G Human insulin;
B(1-29)-B29H-TGLGSGR-A(1-21)-A18K-A21G Human insulin;
B(1-29)-B29A-VGLSSGQ-A(1-21)-A18Q-A22K Human insulin;
B(1-29)-B29A-TGLGSGR-A(1-21)-A18Q-A22K Human insulin;
B(1-29)-B290-TGLGSGR-A(1-21)-A18Q-A22K Human insulin;
B(1-29)-B29H-TGLGSGR-A(1-21)-A18Q-A22K Human insulin;
B(1-29)-B290-TGLGSGR-A(1-21)-A18Q-A21K Human insulin;
B(1-29)-B29Q-TGLGSGR-A(1-21)-A18Q-A21G-A22K Human insulin;
B(1-29)-B29Q-TGLGSGR-A(1-21)-A22G-A23K Human insulin;
B(1-29)-B29A-VGLSSGQ-A(1-21) Human insulin;
B(1-29)-B3K-B29A-VGLSSGQ-A(1-21) Human insulin;
B(1-29)-B22K-B29A-VGLSSGQ-A(1-21) Human insulin;
B(1-29)-B27K-B29A-VGLSSGQ-A(1-21) Human insulin;
B(1-29)-B28K-B29A-VGLSSGQ-A(1-21) Human insulin;
B(1-29)-VGLSSGQ-A(1-21) Human insulin;
B(1-29)-B29A-KGLSSGQ-A(1-21) Human insulin;
B(1-29)-B29A-VKLSSGQ-A(1-21) Human insulin;
B(1-29)-B29A-VGLKSGQ-A(1-21)-A21G Human insulin;
B(1-29)-B29A-TGLGKGQ-A(1-21) Human insulin;
B(1-29)-B29A-VGLSKGQ-A(1-21)-A21G Human insulin;
B(1-29)-B29A-VGLSSGK-A(1-21)-A21G Human insulin;
B(1-29)-B29A-VGLSSGQ-A(1-21)-A8K Human insulin;
B(1-29)-B29A-VGLSSGQ-A(1-21)-A15K Human insulin;
B(1-29)-B29A-VGLSSGQ-A(1-21)-A22K Human insulin;
B(1-29)-B29A-TGLGSGR-A(1-21)-A21G Human insulin;
B(1-29)-B29Q-TGLGSGR-A(1-21)-A21G Human insulin;
B(1-29)-B29H-TGLGSGR-A(1-21)-A21G Human insulin;
B(1-29)-B3K-B29A-TGLGSGR-A(1-21)-A21G Human insulin;
B(1-29)-B3K-B29Q-TGLGSGR-A(1-21)-A21G Human insulin;
B(1-29)-B3K-B29H-TGLGSGR-A(1-21)-A21G Human insulin;
B(1-29)-B20K-B29Q-TGLGSGR-A(1-21)-A21G Human insulin;
B(1-29)-B21K-B29Q-TGLGSGR-A(1-21)-A21G Human insulin;
B(1-29)-B22K-B29H-TGLGSGR-A(1-21)-A21G Human insulin;
B(1-29)-B22K-B29H-TGLGSGR-A(1-21) Human insulin;
B(1-29)-B22K-B29Q-TGLGSGR-A(1-21)-A21G Human insulin;
B(1-29)-B22K-B29Q-TGLGSGR-A(1-21) Human insulin;
B(1-29)-B22K-B29A-TGLGSGR-A(1-21)-A21G Human insulin;
B(1-29)-B22K-B29A-TGLGSGR-A(1-21) Human insulin;
B(1-29)-B22K-B29S-TGLGSGR-A(1-21)-A21G Human insulin;
B(1-29)-B22K-B29S-TGLGSGR-A(1-21) Human insulin;
B(1-29)-B29A-TGLGSGR-A(1-21)-A8K-A21G Human insulin;
B(1-29)-B29Q-TGLGSGR-A(1-21)-A8K-A21G Human insulin;
B(1-29)-B29H-TGLGSGR-A(1-21)-A8K-A18Q-A21G Human insulin;
B(1-29)-B29A-VGLSSGQ-A(1-21)-A22K Human insulin;
B(1-29)-B29A-TGLGSGR-A(1-21)-A22K Human insulin;
B(1-29)-B29Q-TGLGSGR-A(1-21)-A22K Human insulin;
B(1-29)-B29H-TGLGSGR-A(1-21)-A22K Human insulin;
B(1-29)-B29Q-TGLGSGR-A(1-21)-A21K Human insulin;
B(1-29)-B29Q-TGLGSGR-A(1-21)-A21G-A22K Human insulin; and
B(1-29)-B29Q-TGLGSGR-A(1-21)-A18Q-A22G-A23K Human insulin

The parent single-chain insulins are produced by expressing a DNA sequence encoding the single-chain insulin in question in a suitable host cell by well known technique as disclosed in e.g. U.S. Pat. No. 6,500,645. The parent single-chain insulin is either expressed directly or as a precursor molecule which has an N-terminal extension on the B-chain. This N-terminal extension may have the function of increasing the yield of the directly expressed product and may be of up to 15 amino acid residues long. The N-terminal extension is to be cleaved of in vitro after isolation from the culture broth and will therefore have a cleavage site next to B1. N-terminal extensions of the type suitable in the present invention are disclosed in U.S. Pat. No. 5,395,922, and European Patent No. 765,395A.

The polynucleotide sequence coding for the parent single-chain insulin may be prepared synthetically by established standard methods, e.g. the phosphoamidite method described by Beaucage et al. (1981) Tetrahedron Letters 22:1859-1869, or the method described by Matthes et al. (1984) EMBO Journal 3:801-805. According to the phosphoamidite method, oligonucleotides are synthesized, for example, in an automatic DNA synthesizer, purified, duplexed and ligated to form the synthetic DNA construct. A currently preferred way of preparing the DNA construct is by polymerase chain reaction (PCR).

The polynucleotide sequences may also be of mixed genomic, cDNA, and synthetic origin. For example, a genomic or cDNA sequence encoding a leader peptide may be joined to a genomic or cDNA sequence encoding the A and B chains, after which the DNA sequence may be modified at a site by inserting synthetic oligonucleotides encoding the desired amino acid sequence for homologous recombination in accordance with well-known procedures or preferably generating the desired sequence by PCR using suitable oligonucleotides.

The recombinant method will typically make use of a vector which is capable of replicating in the selected microorganism or host cell and which carries a polynucleotide sequence encoding the parent single-chain insulin of the invention. The recombinant vector may be an autonomously replicating vector, i.e., a vector which exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The vector may be linear or closed circular plasmids and will preferably contain an element(s) that permits stable integration of the vector into the host cell's genome or autonomous replication of the vector in the cell independent of the genome.

The recombinant expression vector is capable of replicating in yeast. Examples of sequences which enable the vector to replicate in yeast are the yeast plasmid 2 μm replication genes REP 1-3 and origin of replication.

The vector may contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol or tetracycline resistance. Selectable markers for use in a filamentous fungal host cell include amdS (acetamidase), argB (ornithine carbamoyltransferase), pyrG (orotidine-5′-phosphate decarboxylase) and trpC (anthranilate synthase. Suitable markers for yeast host cells are ADE2, HIS3, LEU2, LYS2, MET3, TRP1, and URA3. A well suited selectable marker for yeast is the Schizosaccharomyces pompe TPI gene (Russell (1985) Gene 40:125-130).

In the vector, the polynucleotide sequence is operably connected to a suitable promoter sequence. The promoter may be any nucleic acid sequence which shows transcriptional activity in the host cell of choice including mutant, truncated, and hybrid promoters, and may be obtained from genes encoding extra-cellular or intra-cellular polypeptides either homologous or heterologous to the host cell.

Examples of suitable promoters for directing the transcription in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), and Bacillus licheniformis penicillinase gene (penP). Examples of suitable promoters for directing the transcription in a filamentous fungal host cell are promoters obtained from the genes for Aspergillus oryzae TAKA amylase, Rhizomucor miehei aspartic proteinase, Aspergillus niger neutral alpha-amylase, and Aspergillus niger acid stable alpha-amylase. In a yeast host, useful promoters are the Saccharomyces cerevisiae Ma1, TPI, ADH or PGK promoters.

The polynucleotide sequence encoding the parent single-chain insulin of the invention will also typically be operably connected to a suitable terminator. In yeast a suitable terminator is the TPI terminator (Alber et al. (1982) J. Mol. Appl. Genet. 1:419-434).

The procedures used to ligate the polynucleotide sequence encoding the parent single-chain insulin of the invention, the promoter and the terminator, respectively, and to insert them into a suitable vector containing the information necessary for replication in the selected host, are well known to persons skilled in the art. It will be understood that the vector may be constructed either by first preparing a DNA construct containing the entire DNA sequence encoding the single-chain insulins of the invention, and subsequently inserting this fragment into a suitable expression vector, or by sequentially inserting DNA fragments containing genetic information for the individual elements (such as the signal, pro-peptide, connecting peptide, A and B chains) followed by ligation.

The vector comprising the polynucleotide sequence encoding the parent single-chain insulin of the invention is introduced into a host cell so that the vector is maintained as a chromosomal integrant or as a self-replicating extra-chromosomal vector. The term “host cell” encompasses any progeny of a parent cell that is not identical to the parent cell due to mutations that occur during replication. The host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, Streptomyces cell, or gram negative bacteria such as E. coli and Pseudomonas sp. Eukaryote cells may be mammalian, insect, plant, or fungal cells. In one embodiment, the host cell is a yeast cell. The yeast organism may be any suitable yeast organism which, on cultivation, produces large amounts of the single chain insulin of the invention. Examples of suitable yeast organisms are strains selected from the yeast species Saccharomyces cerevisiae, Saccharomyces kluyveri, Schizosaccharomyces pombe, Sacchoromyces uvarum, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., and Geotrichum fermentans.

The transformation of the yeast cells may for instance be effected by protoplast formation followed by transformation in a manner known per se. The medium used to cultivate the cells may be any conventional medium suitable for growing yeast organisms. The secreted single-chain insulin, a significant proportion of which will be present in the medium in correctly processed form, may be recovered from the medium by conventional procedures including separating the yeast cells from the medium by centrifugation, filtration or catching the insulin precursor by an ion exchange matrix or by a reverse phase absorption matrix, precipitating the proteinaceous components of the supernatant or filtrate by means of a salt, e.g. ammonium sulphate, followed by purification by a variety of chromatographic procedures, e.g. ion exchange chromatography, affinity chromatography, or the like.

### Pharmaceutical Compositions

The PEGylated single-chain insulins of this invention may be administered subcutaneously, orally, or pulmonary.

For subcutaneous administration, the PEGylated single-chain insulins of this invention are formulated analogously with the formulation of known insulins. Furthermore, for subcutaneous administration, the PEGylated single-chain insulins of this invention are administered analogously with the administration of known insulins and, generally, the physicians are familiar with this procedure.

PEGylated single-chain insulins of this invention may be administered by inhalation in a dose effective to increase circulating insulin levels and/or to lower circulating glucose levels. Such administration can be effective for treating disorders such as diabetes or hyperglycemia. Achieving effective doses of insulin requires administration of an inhaled dose of more than about 0.5 μg/kg to about 50 μg/kg of PEGylated single-chain insulins of this invention. A therapeutically effective amount can be determined by a knowledgeable practitioner, who will take into account factors including insulin level, blood glucose levels, the physical condition of the patient, the patient's pulmonary status, or the like.

According to the invention, the PEGylated single-chain insulins of this invention may be delivered by inhalation to achieve slow absorption thereof. Different inhalation devices typically provide similar pharmacokinetics when similar particle sizes and similar levels of lung deposition are compared.

According to the invention the PEGylated single-chain insulins of this invention may be delivered by any of a variety of inhalation devices known in the art for administration of a therapeutic agent by inhalation. These devices include metered dose inhalers, nebulizers, dry powder generators, sprayers, and the like. Preferably, the PEGylated single-chain insulins of this are delivered by a dry powder inhaler or a sprayer. There are a several desirable features of an inhalation device for administering PEGylated single-chain insulins of this invention. For example, delivery by the inhalation device is advantageously reliable, reproducible, and accurate. The inhalation device should deliver small particles or aerosols, for example, less than about 10 μm, for example about 1-5 μm, for good respirability. Some specific examples of commercially available inhalation devices suitable for the practice of this invention are Turbohaler™ (Astra), Rotahaler® (Glaxo), Diskus® (Glaxo), Spiros™ inhaler (Dura), devices marketed by Inhale Therapeutics, AERx™ (Aradigm), the Ultravent® nebulizer (Mallinckrodt), the Acorn II® nebulizer (Marquest Medical Products), the Ventolin® metered dose inhaler (Glaxo), the Spinhaler® powder inhaler (Fisons), or the like.

As those skilled in the art will recognize, the formulation of PEGylated single-chain insulins this invention, the quantity of the formulation delivered, and the duration of administration of a single dose depend on the type of inhalation device employed. For some aerosol delivery systems, such as nebulizers, the frequency of administration and length of time for which the system is activated will depend mainly on the concentration of PEGylated single-chain insulins in the aerosol. For example, shorter periods of administration can be used at higher concentrations of PEGylated single-chain insulins in the nebulizer solution. Devices such as metered dose inhalers can produce higher aerosol concentrations, and can be operated for shorter periods to deliver the desired amount of the PEGylated single-chain insulins. Devices such as powder inhalers deliver active agent until a given charge of agent is expelled from the device. In this type of inhaler, the amount of insulin PEGylated single-chain insulins of this invention in a given quantity of the powder determines the dose delivered in a single administration.

The particle size of PEGylated single-chain insulins of this invention in the formulation delivered by the inhalation device is critical with respect to the ability of insulin to make it into the lungs, and preferably into the lower airways or alveoli. Preferably, the PEGylated single-chain insulins of this invention ion is formulated so that at least about 10% of the PEGylated single-chain insulins delivered is deposited in the lung, preferably about 10 to about 20%, or more. It is known that the maximum efficiency of pulmonary deposition for mouth breathing humans is obtained with particle sizes of about 2 μm to about 3 μm. When particle sizes are above about 5 μm, pulmonary deposition decreases substantially. Particle sizes below about 1 μm cause pulmonary deposition to decrease, and it becomes difficult to deliver particles with sufficient mass to be therapeutically effective. Thus, particles of the pegylated single-chain insulins delivered by inhalation have a particle size preferably less than about 10 μm, more preferably in the range of about 1 μm to about 5 μm. The formulation of the PEGylated single-chain insulins is selected to yield the desired particle size in the chosen inhalation device.

Advantageously for administration as a dry powder a PEGylated single-chain insulin of this invention is prepared in a particulate form with a particle size of less than about 10 μm, preferably about 1 to about 5 μm. The preferred particle size is effective for delivery to the alveoli of the patient's lung. Preferably, the dry powder is largely composed of particles produced so that a majority of the particles have a size in the desired range. Advantageously, at least about 50% of the dry powder is made of particles having a diameter less than about 10 μm. Such formulations can be achieved by spray drying, milling, or critical point condensation of a solution containing the PEGylated single-chain insulin of this invention and other desired ingredients. Other methods also suitable for generating particles useful in the current invention are known in the art.

The particles are usually separated from a dry powder formulation in a container and then transported into the lung of a patient via a carrier air stream. Typically, in current dry powder inhalers, the force for breaking up the solid is provided solely by the patient's inhalation. In another type of inhaler, air flow generated by the patient's inhalation activates an impeller motor which deagglomerates the particles.

Formulations of PEGylated single-chain insulins of this invention for administration from a dry powder inhaler typically include a finely divided dry powder containing the derivative, but the powder can also include a bulking agent, carrier, excipient, another additive, or the like. Additives can be included in a dry powder formulation of PEGylated single-chain insulin, for example, to dilute the powder as required for delivery from the particular powder inhaler, to facilitate processing of the formulation, to provide advantageous powder properties to the formulation, to facilitate dispersion of the powder from the inhalation device, to stabilize the formulation (for example, antioxidants or buffers), to provide taste to the formulation, or the like. Advantageously, the additive does not adversely affect the patient's airways. The PEGylated single-chain insulin can be mixed with an additive at a molecular level or the solid formulation can include particles of the PEGylated single-chain insulin mixed with or coated on particles of the additive. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; or the like. Typically an additive, such as a bulking agent, is present in an amount effective for a purpose described above, often at about 50% to about 90% by weight of the formulation. Additional agents known in the art for formulation of a protein such as insulin analogue protein can also be included in the formulation.

A spray including the PEGylated single-chain insulins of this invention can be produced by forcing a suspension or solution of the PEGylated single-chain insulin through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of insulin conjugate delivered by a sprayer have a particle size less than about 10 μm, preferably in the range of about 1 μm to about 5 μm.

Formulations of PEGylated single-chain insulins of this invention suitable for use with a sprayer will typically include the PEGylated single-chain insulins in an aqueous solution at a concentration of about 1 mg to about 20 mg of the PEGylated single-chain insulin per ml of solution. The formulation can include agents such as an excipient, a buffer, an isotonicity agent, a preservative, a surfactant, and, preferably, zinc. The formulation can also include an excipient or agent for stabilization of the PEGylated single-chain insulin, such as a buffer, a reducing agent, a bulk protein, or a carbohydrate. Bulk proteins useful in formulating insulin conjugates include albumin, protamine, or the like. Typical carbohydrates useful in formulating the PEGylated single-chain insulin include sucrose, mannitol, lactose, trehalose, glucose, or the like. The PEGylated single-chain insulins formulation can also include a surfactant, which can reduce or prevent surface-induced aggregation of the insulin conjugate caused by atomization of the solution in forming an aerosol. Various conventional surfactants can be employed, such as polyoxyethylene fatty acid esters and alcohols, and polyoxyethylene sorbitol fatty acid esters. Amounts will generally range between about 0.001 and about 4% by weight of the formulation.

Pharmaceutical compositions containing a PEGylated single-chain insulin according to the present invention may also be administered parenterally to patients in need of such a treatment. Parenteral administration may be performed by subcutaneous, intramuscular or intravenous injection by means of a syringe, optionally a pen-like syringe. Alternatively, parenteral administration can be performed by means of an infusion pump.

Injectable compositions of the PEGylated single-chain insulins of the invention can be prepared using the conventional techniques of the pharmaceutical industry which involve dissolving and mixing the ingredients as appropriate to give the desired end product. Thus, according to one procedure, a PEGylated single-chain insulin is dissolved in an amount of water which is somewhat less than the final volume of the composition to be prepared. An isotonic agent, a preservative and a buffer is added as required and the pH value of the solution is adjusted—if necessary—using an acid, e.g. hydrochloric acid, or a base, e.g. aqueous sodium hydroxide as needed. Finally, the volume of the solution is adjusted with water to give the desired concentration of the ingredients.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative which may be selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent which may be selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

Typical isotonic agents are sodium chloride, mannitol, dimethyl sulfone and glycerol and typical preservatives are phenol, m-cresol, methyl p-hydroxybenzoate and benzyl alcohol.

Examples of suitable buffers are sodium acetate, glycylglycine, HEPES (4-(2-hydroxyethyl)-1-piperazineethanesulfonic acid) and sodium phosphate.

A composition for nasal administration of a PEGylated single-chain insulins according to the present invention may, for example, be prepared as described in European Patent No. 272097.

Compositions containing PEGylated single-chain insulins of this invention can be used in the treatment of states which are sensitive to insulin. Thus, they can be used in the treatment of type 1 diabetes, type 2 diabetes and hyperglycaemia for example as sometimes seen in seriously injured persons and persons who have undergone major surgery. The optimal dose level for any patient will depend on a variety of factors including the efficacy of the specific insulin derivative employed, the age, body weight, physical activity, and diet of the patient, on a possible combination with other drugs, and on the severity of the state to be treated. It is recommended that the daily dosage of the PEGylated, single-chain insulin of this invention be determined for each individual patient by those skilled in the art in a similar way as for known insulin compositions.

### DEFINITIONS

With Insulin as used herein is meant human insulin with disulfide bridges between Cys^{A7} and Cys^{B7} and between Cys^{A20} and Cys^{B19} and an internal disulfide bridge between Cys^{A6} and Cys^{A11}, porcine insulin and bovine insulin.

By insulin analogue as used herein is meant a polypeptide which has a molecular structure which formally can be derived from the structure of a naturally occurring insulin, for example that of human insulin, by deleting and/or substituting at least one amino acid residue occurring in the natural insulin and/or by adding at least one amino acid residue. The added and/or substituted amino acid residues can either be codable amino acid residues or other naturally occurring amino acid residues or purely synthetic amino acid residues.

Examples of insulin analogues are such wherein Pro in position 28 of the B chain is mutated with Asp, Lys, or Ile. In another embodiment Lys at position B29 is mutated with Pro, Arg or Ala. Furthermore B27 Thr may be mutated with Arg or Glu. Also, Asn at position A21 may be mutated with Ala, Gln, Glu, Gly, His, Ile, Leu, Met, Ser, Thr, Trp, Tyr or Val, in particular with Gly, Ala, Ser, or Thr and preferably with Gly. Furthermore, Asn at position B3 may be mutated with Thr, Gln, Glu or Asp, and Asn in position A18 may be mutated with Gln. Further examples of insulin analogues are the deletion analogues desBl insulin and desB30 insulin; and insulin analogues wherein the B-chain has an N-terminal extension. Furthermore, the A chain may be extended at its C-terminal end by one or two amino acid residues which are denoted A22 and A23, respectively. Either A22 or A23 may be PEGylated according to the present invention. When the amino acid residue in position A23 is PEGylated then the amino acid in position A22 may be any amino acid residue except Cys and Lys.

By a single-chain insulin is meant a polypeptide sequence of the general structure B-C-A wherein B is the human B insulin chain or an analogue or derivative thereof, A is the human insulin A chain or an analogue or derivative and C is a peptide chain of 3-35 amino acid residues connecting the C-terminal amino acid residue in the B-chain with A1. If the B chain is a desB30 chain the connecting peptide will connect B29 with A1, The single-chain insulin will contain correctly positioned disulphide bridges (three) as in human insulin that is between CysA7 and CysB7 and between CysA20 and CysB19 and an internal disulfide bridge between CysA6 and CysA11.

Analogues of the B and A chains of the human insulin B and A chains are insulin B and A chains having one or more mutations, substitutions, deletions and or additions of the A and/or B amino acid chains relative to the human insulin molecule.

The term analogue as used herein referring to a peptide means a modified peptide wherein one or more amino acid residues of the peptide have been substituted by other amino acid residues and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. In one embodiment an analogue comprises less than 5 modifications (substitutions, deletions, additions) relative to the native peptide. In another embodiment an analogue comprises less than 4 modifications (substitutions, deletions, additions) relative to the native peptide. In another embodiment an analogue comprises less than 3 modifications (substitutions, deletions, additions) relative to the native peptide. In another embodiment an analogue comprises less than 2 modifications (substitutions, deletions, additions) relative to the native peptide. In another embodiment an analogue comprises only a single modification (substitutions, deletions, additions) relative to the native peptide.

With desB30 or B(1-29) is meant a natural insulin B chain or an analogue thereof lacking the B30 amino acid residue, A(1-21) means the natural insulin A chain or an analogue or derivative thereof. The amino acid residues are indicated in the three letter amino acid code or the one letter amino code.

With B1, A1 etc. is meant the position 1 in the B chain of insulin (counted from the N-terminal end) and the position 1 in the A chain of insulin (counted from the N-terminal end), respectively.

With fast acting insulin is meant an insulin having a faster onset of action than normal or regular human insulin.

With long acting insulin is meant an insulin having a longer duration of action than normal or regular human insulin.

With connecting peptide is meant a peptide chain which connects the C-terminal amino acid residue of the B-chain with the N-terminal amino acid residue of the A-chain.

The term basal insulin as used herein means an insulin peptide which has a time-action of more than 8 hours, in particularly of at least 9 hours. Preferably, the basal insulin has a time-action of at least 10 hours. The basal insulin may thus have a time-action in the range from 9 to 15 hours.

With parent insulin is meant the single-chain insulin peptide back bone chain with the modifications in the amino acid residue composition according to the present invention.

With “PEG” or polyethylene glycol, as used herein is meant any water soluble poly(alkylene oxide). The expression PEG will cover the structure —CH₂CH₂O(CH₂CH₂O)ₙCH₂CH₂O— where n is an integer from 2 to about 600. A commonly used PEG is end-capped PEG, wherein one end of the PEG is capped with a relatively inactive group such as an alkoxy while the other end is a hydroxyl group that may be further modified. An often used capping group is methoxy and the corresponding end-capped PEG is often denoted mPEG. The notion PEG is often used instead of mPEG.

Specific PEG forms of the invention is branched, linear, forked PEGs, and the like and the PEG groups are typically polydisperse, possessing a low polydispersity index of less than about 1.05.

The PEG moieties of the invention will for a given molecular weight will typically consist of a range of ethyleneglycol (or ethyleneoxide) monomers. For example, A PEG moiety of molecular weight 2000 will typically consist of 43±10 monomers, the average being around 43 monomers.

By PEGylated single-chain insulin having insulin activity is meant a PEGylated, single-chain insulin with the ability to lower the blood glucose in mammalians as measured in a suitable animal model, which may be a rat, rabbit, or pig model, after suitable administration e.g. by intravenous or subcutaneous administration.

By high physical stability is meant a tendency to fibrillation being less than 50% of that of human insulin. Fibrillation may be described by the lag time before fibril formation is initiated at a given conditions.

A polypeptide with Insulin receptor and IGF-1 receptor affinity is a polypeptide which is capable of interacting with an insulin receptor and a human IGF-1 receptor in a suitable binding assay. Such receptor assays are well-know within the field and are further described in the examples. The present PEGylated single-chain insulin will not bind to the IGF-1 receptor or will have a rather low affinity to said receptor. More precisely the present PEGylated single-chain insulins will have an affinity towards the IGF-1 receptor of substantially the same magnitude or less as that of human insulin

The terms treatment and treating as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

The term treatment of a disease as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

The term prevention of a disease as used herein is defined as the management and care of an individual at risk of developing the disease prior to the clinical onset of the disease. The purpose of prevention is to combat the development of the disease, condition or disorder, and includes the administration of the active compounds to prevent or delay the onset of the symptoms or complications and to prevent or delay the development of related diseases, conditions or disorders.

The term effective amount as used herein means a dosage which is sufficient in order for the treatment of the patient to be effective compared with no treatment.

POT is the Schizosaccharomyces pombe triose phosphate isomerase gene, and TPI1 is the S. cerevisiae triose phosphate isomerase gene.

By a leader is meant an amino acid sequence consisting of a pre-peptide (the signal peptide) and a pro-peptide.

The term signal peptide is understood to mean a pre-peptide which is present as an N-terminal sequence on the precursor form of a protein. The function of the signal peptide is to allow the heterologous protein to facilitate translocation into the endoplasmic reticulum. The signal peptide is normally cleaved off in the course of this process. The signal peptide may be heterologous or homologous to the yeast organism producing the protein. A number of signal peptides which may be used with the DNA construct of the invention including yeast aspartic protease 3 (YAP3) signal peptide or any functional analog (Egel-Mitani et al. (1990) YEAST 6:127-137 and U.S. Pat. No. 5,726,038) and the α-factor signal of the MFα1 gene (Thorner (1981) in The Molecular Biology of the Yeast Saccharomyces cerevisiae, Strathern et al., eds., pp 143-180, Cold Spring Harbor Laboratory, NY and U.S. Pat. No. 4,870,00.

The term pro-peptide means a polypeptide sequence whose function is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The pro-peptide may be the yeast a-factor pro-peptide, vide U.S. Pat. Nos. 4,546,082 and 4,870,008. Alternatively, the pro-peptide may be a synthetic pro-peptide, which is to say a pro-peptide not found in nature. Suitable synthetic pro-peptides are those disclosed in U.S. Pat. Nos. 5,395,922; 5,795,746; 5,162,498 and WO 98/32867. The pro-peptide will preferably contain an endopeptidase processing site at the C-terminal end, such as a Lys-Arg sequence or any functional analogue thereof.

In the present context the three-letter or one-letter indications of the amino acids have been used in their conventional meaning as indicated in the following. Unless indicated explicitly, the amino acids mentioned herein are L-amino acids. Further, the left and right ends of an amino acid sequence of a peptide are, respectively, the N- and C-termini unless otherwise specified.

Abbreviations for Amino Acids

| Amino acid | Three-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

The following abbreviations have been used in the specification and examples:
Bzl=Bn: benzyl

### DIEA: N, N-diisopropylethylamine

### DMF: N,N-dimethylformamide

tBu: tert-butyl
Glu: Glutamic acid
TSTU: O-(N-succinimidyl)-1,1,3,3-tetramethyluronium tetrafluoroborate

### THF: Tetrahydrofuran

EtOAc: Ethyl acetate

### DIPEA: Diisopropylethylamine (=DIEA)

HOAt: 1-Hydroxy-7-azabenzotriazole

### NMP: N-methylpyrrolidin-2-one

TEA: triethyl amine
SA: Sinapinic acid
Su: 1-succinimidyl=2,5-dioxo-pyrrolidin-1-yl
TFA: trifluoracetic acid
DCM: dichloromethane
DMSO: dimethyl sulphoxide
RT: room temperature
mPEG-SPA is mPEG-CH₂CH₂—CO—OSu (N-hydroxysuccinimidyl ester of mPEG-propionic acid);
mPEG-SBA is mPEG-CH₂CH₂CH₂—CO—OSu (N-hydroxysuccinimidyl ester of mPEG-butanoic acid);
mPEG-SMB is mPEG-CH₂CH₂CH(CH₃)—CO—OSu (N-hydroxysuccinimidyl ester of mPEG-α-methylbutanoic acid;
mPEG is CH₃O(CH₂CH₂O)ₙCH₂CH₂—O—, where n is an integer from 2 to 600 sufficient to give the average molecular weight indicated for the whole PEG moiety, eg for mPEG Mw 2.000, n is approximately 43.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

### General Procedures

The following examples and general procedures refer to intermediate compounds and final products identified in the specification. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents.

The compounds of the invention can be purified by employing one or more of the following procedures which are typical within the art. These procedures can—if needed—be modified with regard to gradients, pH, salts, concentrations, flow, columns and so forth. Depending on factors such as impurity profile, solubility of the insulins in question etcetera, these modifications can readily be recognised and made by a person skilled in the art.

After acidic HPLC or desalting, the compounds are isolated by lyophilisation of the pure fractions.

After neutral HPLC or anion exchange chromatography, the compounds are desalted, precipitated at isoelectric pH, or purified by acidic HPLC.

### Typical Purification Procedures:

The HPLC system is a Gilson system consisting of the following: Model 215 Liquid handler, Model 322-H2 Pump and a Model 155 UV Dector. Detection is typically at 210 nm and 280 nm.
The Âkta Purifier FPLC system (Amersham Biosciences) consists of the following: Model P-900 Pump, Model UV-900 UV detector, Model pH/C-900 pH and conductivity detector, Model Frac-950 Frction collector. UV detection is typically at 214 nm, 254 nm and 276 nm.

### Acidic HPLC:

| Column: | Macherey-Nagel SP 250/21 Nucleosil 300-7 C4 | |
|---|---|---|
| Flow: | 8 ml/min, | |
| Buffer A: | 0.1% TFA in acetonitrile | |
| Buffer B: | 0.1% TFA in water. | |
| Gradient: | 0.0-5.0 min: | 10% A |
| | 5.00-30.0 min: | 10% A to 90% A |
| | 30.0-35.0 min: | 90% A |
| | 35.0-40.0 min: | 100% A |

### Neutral HPLC:

| Column: | Phenomenex, Jupiter, C4 5 μm 250 × 10.00 mm, 300 Å | |
|---|---|---|
| Flow: | 6 ml/min | |
| Buffer A: | 5 mM TRIS, 7.5 mM (NH₄)₂SO₄, pH = 7.3, 20% CH₃CN | |
| Buffer B: | 60% CH₃CN, 40% water | |
| Gradient: | 0-5 min: | 10% B, |
| | 5-35 min: | 10-60% B |
| | 35-39 min: | 60% B, |
| | 39-40 min: | 70% B |
| | 40-43.5 min: | 70% B |

### Anion Exchange Chromatography:

| | |
|---|---|
| Column: | RessourceQ, 1 ml |
| Flow: | 6 ml/min |
| Buffer A: | 0.09% NH₄HCO₃, 0.25% NH₄OAc, 42.5% ethanol pH 8.4 |
| Buffer B: | 0.09% NH₄HCO₃, 2.5% NH₄OAc, 42.5% ethanol pH 8.4 |
| Gradient: | 100% A to 100% B during 30 column volumes |

### Desalting:

| | |
|---|---|
| Column: | HiPrep 26/10 |
| Flow: | 10 ml/min, 6 column volumes |
| Buffer: | 10 mM NH₄HCO₃ |

The following analytical HPLC systems were used:

### Method 1:

Two Waters 510 HPLC pumps
Waters 2487 Dual λ Absorbance detector

| | |
|---|---|
| Run time: | 30 min. |
| Injection: | 25 μl. |
| Buffer A: | 0.1% TFA in acetonitrile. |
| Buffer B: | 0.1% TFA in water. |
| Flow: | 1.5 ml/min. |
| Gradient: | 1-17 min: 25% B to 85% B, 17-22 min: 85% B, 22-23 min: |
| | 85% B to 25% B, 23-30 min 25% B, 30-31 min 25% B flow: |
| | 0.15 ml/min. |
| Column: | C4 5μ 150 × 4_60 mm “phenomenex, Jupiter”. |
| Detection: | UV 214 nm. |

### Method 2:

Two Waters 510 HPLC pumps
Waters 2487 Dual λ Absorbance detector

| | |
|---|---|
| Run time: | 30 min. |
| Injection: | 25 μl. |
| Buffer A: | 0.1% TFA, 10% CH₃CN, 89.9% water. |
| Buffer B: | 0.1% TFA, 80% CH₃CN, 19.9% water. |
| Flow: | 1.5 ml/min. |
| Gradient: | 0-17 min: 20%-90% B, 17-21 min 90% B. |
| Column: | C4 5μ 150 × 4_60 mm “phenomenex, Jupiter”. |
| Detection: | UV 214 nm. |

### Method 3:

Two Waters 510 HPLC pumps
Waters 486 Tunable Absorbance Detector
Waters 717 Autosampler

| | |
|---|---|
| Column: | C4 5μ 150 × 4_60 mm “phenomenex, Jupiter”. |
| Injection: | 20 μl. |
| Buffer A: | 80% 0.0125 M Tris, 0.0187 M (NH₄)₂SO₄ pH = 7, 20% |
| | CH₃CN. |
| Buffer B: | 80% CH₃CN, 20% water. |
| Flow: | 1.5 ml/min. |
| Gradient: | 0 min 5% B −> 20 min 55% B −> 22 min 80% B −> 24 min |
| | 80% B −> 25 min 5% B 32 min 5% B. |
| Detection: | UV 214 nm. |

### Method 4:

Two Waters 510 HPLC pumps
Waters 2487 Dual λ Absorbance detector

| | |
|---|---|
| Column: | C4 5μ 150 × 4_60 mm “phenomenex, Jupiter” |
| Injection: | 20 μl |
| Buffer A: | 80% 0.0125 M Tris, 0.0187 M (NH₄)₂SO₄ pH = 7, 20% |
| | CH₃CN |
| Buffer B: | 80% CH₃CN, 20% water |
| Flow: | 1.5 ml/min |
| Gradient: | 0 min 10% B −>20 min 50% B −> 22 min 60% B −> 23 min |
| | 10% B −> 30 min 10% B −> 31 min 10% B flow 0.15 min |
| Detection: | 214 nm |

### Method 5:

Waters 2695 separations module
Waters 996 Photodiode Array Detector

| | |
|---|---|
| Column: | C4 5μ 150 × 4_60 mm “phenomenex, Jupiter” |
| Injection: | 25 μl |
| Buffer A: | 80% 0.01 M Tris, 0.015 M (NH₄)₂SO₄ pH = 7.3; 20% |
| | CH₃CN |
| Buffer B: | 20% water; 80% CH₃CN |
| Flow: | 1.5 ml/min |
| Gradient: | 1-20 min: 5-50% B, 20-22 min: 50-60% B, 22-23 min: |
| | 60-5% B, 23-30 min 5-0% B 30-31 min 0-5% B, flow: |
| | 0.15 ml/min. |
| Detection: | 214 nm |

### Method 6:

Waters 2795 separations module

### Waters 2996 Photodiode Array Detector

Waters Micromass ZQ 4000 electrospray mass spectrometer

### LC-Method:

| | |
|---|---|
| Column: | Phenomenex, Jupiter 5μ C4 300 Å 50 × 4.60 mm |
| Buffer A: | 0.1% TFA in water |
| Buffer B: | CH₃CN |
| Flow: | 1 ml/min |
| Gradient: | 0-7.5 min: 10-90% B |
| | 7.5-8.5 min: 90-10% B |
| | 8.5-9.5 min 10% B |
| | 9.5-10.00 min 10% B, flow: 0.1 ml/min |
| MS method: | Mw: 500-2000 ES+ |
| | Cone Voltage 60 V |
| | Scantime 1 |
| | InterScan delay: 0.1 |

### Method 7:

Agilent 1100 series

| | |
|---|---|
| Column: | GraceVydac Protein C4, 5 um 4.6 × 250 mm (Cat# |
| | 214TP54) |
| Buffer A: | 10 mM Tris, 15 mM (NH₄)₂SO₄, 20% CH₃CN in water |
| | pH 7.3 |
| Buffer B: | 20% water in CH₃CN |
| Flow: | 1.5 ml/min |
| Gradient: | 1-20 min: 10% B to 50% B, 20-22 min: 50% B to 60% B, |
| | 22-23 min: 60% B to 10% B, 23-30 min 10% B 30-31 min |
| | 10% B, flow 0.15 ml/min. |
| Detection: | 214 nm. |

MALDI-TOF-MS spectra were recorded on a Bruker Autoflex II TOF/TOF operating in linear mode using a nitrogen laser and positive ion detection. Accelerating voltage: 20 kV.

In the following list, selected PEGylation reagents are listed as activated N-hydroxysuccinimide esters (OSu). Obviously other active esters may be employed, such as 4-nitrophenoxy and many other active esters known to those skilled in the art. The PEG (or mPEG) moiety, CH₃O—(CH₂CH₂O)ₙ—, can be of any size up to Mw 40.000 Da. The structure/sequence of the PEG-residue on the single-chain insulin can formally be obtained by replacing the leaving group (eg. “—OSu”) from the various PEGylation reagents with “NH-single chain insulin”, where the single chain insulin is PEGylated either in an epsilon position in a lysine residue or in the alpha-amino position in the B-chain (or both):

mPEG-COCH₂CH₂CO—OSu
mPEG-COCH₂CH₂CH₂CO—OSu
mPEG-CH₂CO—OSu
mPEG-CH₂CH₂CO—OSu
mPEG-CH₂CH₂CH₂CO—OSu
mPEG-CH₂CH₂CH₂CH₂CO—OSu
mPEG-CH₂CH₂CH₂CH₂CH₂CO—OSu
mPEG-CH₂CH(CH₃)CO—OSu
mPEG-CH₂CH₂CH(CH₃)CO—OSu
mPEG-CH₂CH₂NH—COCH₂CH₂CO—OSu
mPEG-CH₂CH₂CH₂NH—COCH₂CH₂CH₂CO—OSu
mPEG-CH₂CH₂CH₂NH—COCH₂CH₂CO—OSu
mPEG-CH₂CH₂NH—COCH₂CH₂CH₂CO—OSu
mPEG-CO-(4-nitrophenoxy)

Recombinant Methods:

All expressions plasmids are of the C—POT type, similar to those described in EP 171, 142, which are characterized by containing the Schizosaccharomyces pombe triose phosphate isomerase gene (POT) for the purpose of plasmid selection and stabilization in S. cerevisiae. The plasmids also contain the S. cerevisiae triose phosphate isomerase promoter and terminator. These sequences are similar to the corresponding sequences in plasmid pKFN1003 (described in WO 90/100075) as are all sequences except the sequence of the EcoRI-XbaI fragment encoding the fusion protein of the leader and the insulin product. In order to express different fusion proteins, the EcoRI-XbaI fragment of pKFN1003 is simply replaced by an EcoRI-XbaI fragment encoding the leader-insulin fusion of interest. Such EcoRI-XbaI fragments may be synthesized using synthetic oligonucleotides and PCR according to standard techniques.

Yeast transformants were prepared by transformation of the host strain S. cerevisiae strain MT663 (MATa/MATα pep4-3/pep4-3 HIS4/his4 tpi::LEU2/tpi::LEU2 cir⁺). The yeast strain MT663 was deposited in the Deutsche Sammlung von Mikroorganismen and Zellkulturen in connection with filing WO 92/11378 and was given the deposit number DSM 6278.

MT663 was grown on YPGaL (1% Bacto yeast extract, 2% Bacto peptone, 2% galactose, 1% lactate) to an O.D. at 600 nm of 0.6. 100 ml of culture was harvested by centrifugation, washed with 10 ml of water, recentrifuged and resuspended in 10 ml of a solution containing 1.2 M sorbitol, 25 mM Na₂EDTA pH=8.0 and 6.7 mg/ml dithiotreitol. The suspension was incubated at 30° C. for 15 minutes, centrifuged and the cells resuspended in 10 ml of a solution containing 1.2 M sorbitol, 10 mM Na₂EDTA, 0.1 M sodium citrate, pH 0 5.8, and 2 mg Novozym®234. The suspension was incubated at 30° C. for 30 minutes, the cells collected by centrifugation, washed in 10 ml of 1.2 M sorbitol and 10 ml of CAS (1.2 M sorbitol, 10 mM CaCl₂, 10 mM Tris HCl (Tris=Tris(hydroxymethyl)aminomethane) pH=7.5) and resuspended in 2 ml of CAS. For transformation, 1 ml of CAS-suspended cells was mixed with approx. 0.1 mg of plasmid DNA and left at room temperature for 15 minutes. 1 ml of (20% polyethylene glycol 4000, 10 mM CaCl₂, 10 mM Tris HCl, pH=7.5) was added and the mixture left for a further 30 minutes at room temperature. The mixture was centrifuged and the pellet resuspended in 0.1 ml of SOS (1.2 M sorbitol, 33% v/v YPD, 6.7 mM CaCl₂) and incubated at 30° C. for 2 hours. The suspension was then centrifuged and the pellet resuspended in 0.5 ml of 1.2 M sorbitol. Then, 6 ml of top agar (the SC medium of Sherman et al. (1982) Methods in Yeast Genetics, Cold Spring Harbor Laboratory) containing 1.2 M sorbitol plus 2.5% agar) at 52° C. was added and the suspension poured on top of plates containing the same agar-solidified, sorbitol containing medium.

S. cerevisiae strain MT663 transformed with expression plasmids was grown in YPD for 72 h at 30° C.

### Example 1

### B(1-29)-B3Q-B29R-TGLGK(N^{ε}-3-(mPEG2000-yl)propionyl)GQ-A(1-21)-A18Q-A21G Human Insulin

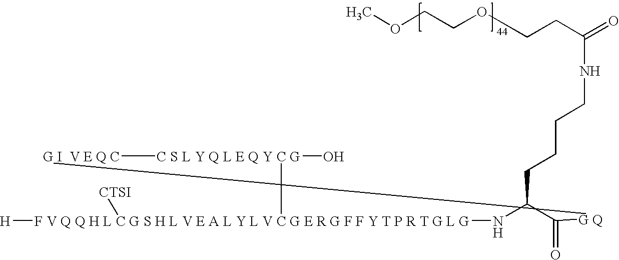

B(1-29)-B3Q-B29R-TGLGKGQ-A(1-21)-A18Q-A21G Human insulin (90 mg, 14 μmol) was dissolved in 100 mM aqueous Na₂CO₃ (1 ml). A solution of mPEG-SPA (Nektar, Mw 2.000 Da) (28 mg; 14 μmol) in acetonitrile (1 ml) was then added followed by more 100 mM aqueous Na₂CO₃ (0.8 ml). The reaction mixture (pH 10-11) was stirred gently at room temperature for 45 min, then pH was adjusted to 5.2 with 1M aqueous HCl. The mixture was purified by preparative HPLC using a Macherey-Nagel SP 250/21 Nucleusil 300-7 C4 column eluting with a linear gradient of 25% to 90% buffer B. Buffer A: 0.1% TFA in MiliQ water, buffer B: 0.1% TFA in acetonitrile. Fractions were then analyzed individually using LC-MS and MALDI-TOF. Fractions containing pure product was pooled, diluted with water and lyophilised to give 5 mg of title material. Further material can be obtained by purification of impure fractions (44 mg).

HPLC (Method 2): Rt=10.25 min, 100% purity

MALDI-TOF-MS (matrix: sinapinic acid (SA)); m/z≈8600

### Example 2

### B(1-29)-B22K(N^{ε}-3-(mPEG2000-yl)propionyl)-B29A-A18Q-VGLSSGQ-A(1-21) Human Insulin

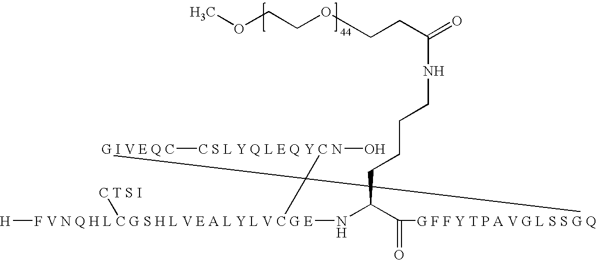

B(1-29)-B22K-B29A-A18Q-VGLSSGQ-A(1-21) Human insulin (70 mg, 11 μmol) was dissolved in 100 mM aqueous Na₂CO₃ (2.3 ml). A solution of mPEG-SPA (Nektar, Mw 2.000 Da) (22 mg; 11 μmol) in acetonitrile (1.1 ml) was then added. The reaction mixture (pH 10-11) was stirred gently at room temperature for 1 hour, then pH was adjusted to 5.6 with 1M aqueous HCl. The mixture was purified by preparative HPLC using a Macherey-Nagel SP 250/21 Nucleusil 300-7 C4 column eluting with a linear gradient of 20% to 90% buffer B. Buffer A: 0.1% TFA in MiliQ water, buffer B: 0.1% TFA in acetonitrile. Fractions were then analyzed individually using LC-MS and MALDI-TOF. Fractions containing pure product was pooled, diluted with water and lyophilised to give 13 mg of title material.

HPLC (Method 1): Rt=9.76 min, 99.9% purity.

HPLC (Method 3): Rt=10.34 min, 99.6% purity

MALDI-TOF-MS (SA): m/z≈8300:

### Example 3

### B(1-29)-B29K(N^{ε}-3-(mPEG2000-yl-propionyl)-VGLSSGQ-A(1-21)-A18Q Human Insulin

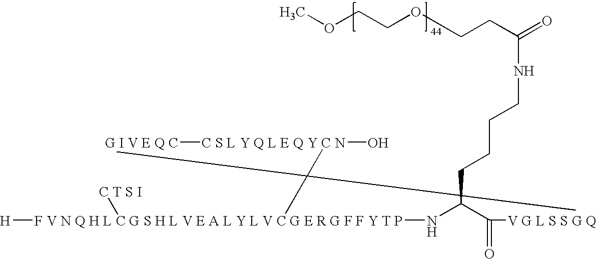

B(1-29)-VGLSSGQ-A(1-21)-A18Q Human insulin (400 mg, 63 μmol) was dissolved in 0.1 M Na₂CO₃ (6 ml) and a solution of mPEG-SPA (Nektar, Mw 2.000 Da) (135 mg) in acetonitrile (6 ml) was added, pH was adjusted to 10.3 with a few drops of 1N sodium hydroxide. The mixture was stirred gently for 70 minutes and pH was adjusted to 5.3 using 1 N hydrochloric acid. The organic solvent was removed by evaporation in vacuo and the residue was lyophilised. The crude product was re-dissolved in a mixture of water and acetonitrile and purified in several runs by preparative HPLC using a Macherey-Nagel SP 250/21 Nucleusil 300-7 C4 column eluting with a linear gradient of 20% to 80% buffer B. Buffer A: 0.1% TFA in MiliQ water, buffer B: 0.1% TFA in acetonitrile.

Fractions were then analyzed individually using LC-MS and MALDI-TOF. Fractions containing pure product was pooled, diluted with water and lyophilised to give 302 mg of title material.

HPLC (Method 1): Rt=9.66 min, 100% purity.

HPLC(Method 3): Rt=10.03 min, 97.2% purity

MALDI-TOF-MS (SA); m/z≈8600:

The following examples were prepared similarly:

### Example 4

### B(1-29)-B29A-VGLSSGQ-A(1-21)-A8K(N^{ε}-3-(mPEG2000-yl)propionyl)-A18Q Human Insulin

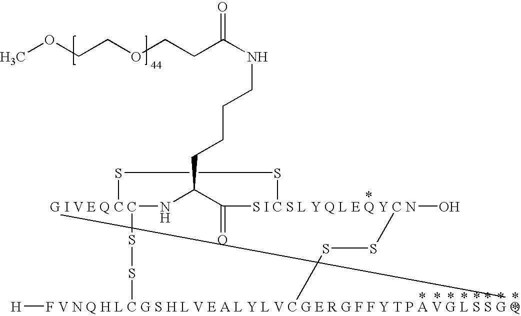

MALDI-TOF-MS (matrix: SA): m/z=8200-8750

HPLC (Method 1): Rₜ=10.58 min

HPLC (Method 5): Rₜ=9.83 min

### Example 5

### B(1-29)-B29A-VK(N^{ε}-3-(mPEG2000-yl)propionyl)LSSGQ-A(1-21)-A18Q Human Insulin

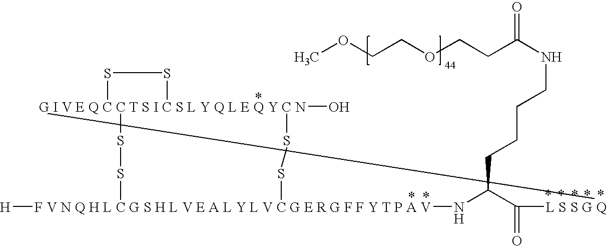

MALDI-TOF-MS (matrix: cyano): m/z centered around 8598

HPLC (Method 1): Rₜ=8.94 min

HPLC (Method 5): Rₜ=10.23 min

### Example 6

### B(1-29)-B29A-VGLSSGQ-A(1-22)-A18Q-A22K(N^{ε}-3-(mPEG2000-yl)propionyl) Human Insulin

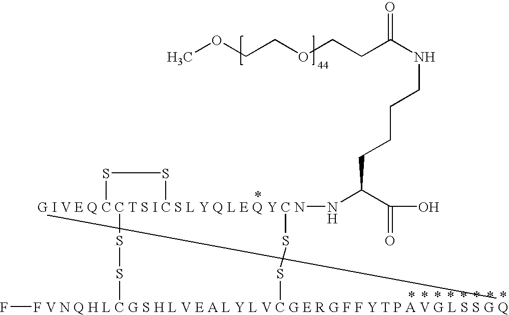

MALDI-TOF-MS (matrix: cyano) m/z: between 8159 and 9085

HPLC (Method 1): Rₜ=8.86 min

HPLC (Method 5): Rₜ=9.56 min

HPLC (Method 6): Rₜ=9.24 min.

### Example 7

### B(1-29)-B3K(N^{ε}-3-(mPEG2000-yl)propionyl)-B29A-VGLSSGQ-A(1-21)-A18Q Human Insulin

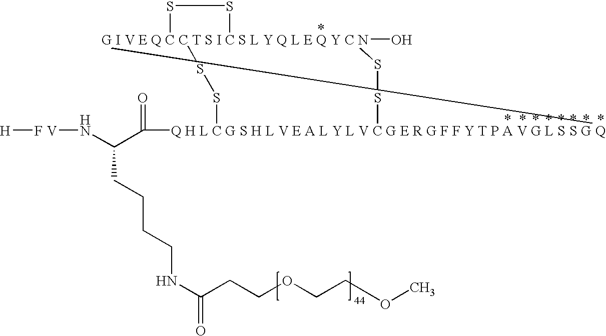

MALDI-TOF-MS (matrix: cyano): m/z: centered around 8532.

HPLC (Method 1): Rₜ=14.08 min

HPLC (Method 5): Rₜ=8.11 min

HPLC (Method 6): Rₜ=9.13 min.

### Example 8

### B(1-29)-B1(N^{α}-3-(mPEG2000-yl)propionyl)-B29A-VGLSSGQ-A(1-21)-A18Q Human Insulin

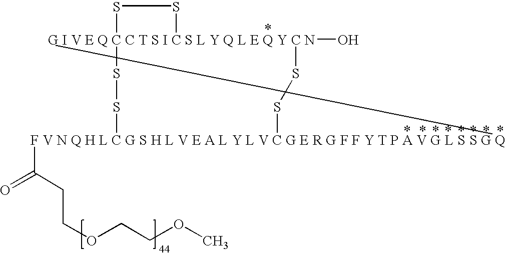

HPLC (Method 1): Rₜ=9.38 min

HPLC (Method 5): Rₜ=8.47 min

The PEGylated single-chain insulins were tested for biological insulin activity as measured by binding affinity to the human insulin receptor (IR) relative to that of human insulin as described below. The results are shown in the following table.

| | IR binding in percent |
|---|---|
| | of human insulin |
| Example 1 | 6.8% |
| Example 2 | 19.7% |
| Example 3 | 5.3% |
| Example 4 | 4.6% |
| Example 5 | 3.2% |
| Example 6 | 12% |
| Example 7 | 4.5% |
| Example 8 | 8.1% |

Pharmacological Methods

### Assay (I)

Insulin receptor binding of the PEGylated single-chain insulins of the invention.

The affinity of the PEGylated, single-chain insulins the invention for the human insulin receptor is determined by a SPA assay (Scintillation Proximity Assay) microtiterplate antibody capture assay. SPA-PVT antibody-binding beads, anti-mouse reagent (Amersham Biosciences, Cat No. PRNQ0017) are mixed with 25 ml of binding buffer (100 mM HEPES pH 7.8; 100 mM sodium chloride, 10 mM MgSO4, 0.025% Tween-20). Reagent mix for a single Packard Optiplate (Packard No. 6005190) is composed of 2.4 μl of a 1:5000 diluted purified recombinant human insulin receptor—exon 11, an amount of a stock solution of A14 Tyr[125I]-human insulin corresponding to 5000 cpm per 100 μl of reagent mix, 12 μl of a 1:1000 dilution of F12 antibody, 3 ml of SPA-beads and binding buffer to a total of 12 ml. A total of 100 μl is then added and a dilution series is made from appropriate samples. To the dilution series is then added 100 μl of reagent mix and the samples were incubated for 16 hours while gently shaken. The phases are then separated by centrifugation for 1 min and the plates counted in a Topcounter. The binding data are fitted using the nonlinear regression algorithm in the GraphPad Prism 2.01 (GraphPad Software, San Diego, Calif.).

### Assay (II)

Alternatively the insulin receptor binding is tested in a hIRBHK membrane assay as follows:

Binding of [¹²⁵I]-human insulin to membrane-associated recombinant human insulin receptor isoform A (hIR-A)

### Reagents:

¹²⁵I-Insulin: Novo Nordisk A/S, mono ¹²⁵I-(Tyr A14) human insulin

### Human Insulin: Novo Nordisk A/S,

Human serum albumin: Dade Behring, ORHA 194 C30, lot 455077
Plastic ware: Packard OptiPlate™-96, #6,005,290
Scintillant: Amersham Biosciences, WGA coated PVT microspheres, # RPNQ0001
Cells: BHK tk⁻ ts13 cells expressing recombinant human insulin receptor isoform A (hIR12-14).

Extraction of membrane-associated insulin receptors: BHK cells from a ten-layer cell factory are harvested and homogenised in 25 ml of ice-cold buffer (25 mM HEPES pH 7.4, 2.5 mM CaCl₂, 1 mM MgCl₂, 250 mg/l bacitracin, 0.1 mM Pefablock). The homogenate is layered carefully on 41% sucrose cushions, centrifuged in the ultracentrifuge at 95,000×g for 75 minutes in a Beckman SW28 rotor at 4° C. The plasma membranes are collected from the top of the sucrose cushion, diluted 1:4 with buffer and centrifuged at 40,000×g for 45 min in a Beckman SW28 rotor. The pellets are suspended in buffer (25 mM HEPES pH 7.4, 2.5 mM CaCl₂, 1 mM MgCl₂, 250 mg/l bacitracin, 0.1 mM Pefablock) and stored at −80° C.

Radioligand binding to membrane-associated insulin receptors is performed in duplicate in 96-well OptiPlates. Membrane protein is incubated for 150 minutes at 25° C. with 50 pM [¹²⁵I-Tyr^{A14}]-human insulin in a total volume of 200 ml assay buffer (50 mM HEPES, 150 mM NaCl, 5 mM MgSO₄, 0.01% Triton X-100, 0.1% HSA, Complete™ EDTA-free protease inhibitors) and increasing concentrations of human insulin or insulin analogues (typically between 0.01 and 300 nM). The assay is terminated by addition of 50 μl of a suspension of WGA-coated PVT microspheres (20 mg/ml). Following 5 minutes of slight agitation, the plate is centrifuged at 1500 RPM for 6 minutes, and bound radioactivity quantified by counting in a Packard TopCount NXT after a delay of 60 minutes.

Results are given as IC₅₀ relative to human insulin in %.

### Assay (III)

Potency of the PEGylaed, single-chain insulins relative to human insulin.

Wistar rats are used for testing the blood glucose lower efficacy of SCI of I.V bolus administration. Following administration the of either SCI or human insulin the concentration of blood glucose is monitored

### Assay (IV)

Determination in pigs of T50% of the PEGylated, single-chain insulins

T50% is the time when 50% of an injected amount of the A14 Tyr[125I] labelled derivative of an insulin to be tested has disappeared from the injection site as measured with an external γ-counter.

The principles of laboratory animal care are followed, Specific pathogen-free LYYD, non-diabetic female pigs, cross-breed of Danish Landrace, Yorkshire and Duroc, are used (Holmenlund, Haarloev, Denmark) for pharmacokinetic and pharmacodynamic studies. The pigs are conscious, 4-5 months of age and weighing 70-95 kg. The animals are fasted overnight for 18 h before the experiment.

Formulated preparations of insulin derivatives labelled in TyrA14 with 125I are injected sc. in pigs as previously described (Ribel, U., Jørgensen, K, Brange, J, and Henriksen, U. The pig as a model for subcutaneous insulin absorption in man. Serrano-Rios, M and Lefèbvre, P. J. 891-896. 1985. Amsterdam; New York; Oxford, Elsevier Science Publishers. 1985 (Conference Proceeding)).

At the beginning of the experiments a dose of 60 nmol of the insulin test compound and a dose of 60 nmol of insulin (both 125I labelled in Tyr A14) are injected at two separate sites in the neck of each pig.

The disappearance of the radioactive label from the site of sc. Injection is monitored using a modification of the traditional external gamma-counting method (Ribel, U. Subcutaneous absorption of insulin analogues. Berger, M. and Gries, F. A. 70-77 (1993). Stuttgart; New York, Georg Thime Verlag (Conference Proceeding)). With this modified method it is possible to measure continuously the disappearance of radioactivity from a subcutaneous depot for several days using cordless portable device (Scancys Laboratorieteknik, Værløse, DK-3500, Denmark). The measurements are performed at 1-min intervals, and the counted values are corrected for background activity.

### Assay (V)

Pulmonary Delivery to Rats The test substance will be dosed pulmonary by the drop instillation method. In brief, male Wistar rats (app. 250 g) are anaesthetized in app. 60 ml fentanyl/dehydrodenzperidol/-dormicum given as a 6.6 ml/kg sc priming dose and followed by 3 maintenance doses of 3.3 ml/kg sc with an interval of 30 min. Ten minutes after the induction of anaesthesia, basal samples are obtained from the tail vein (t=−20 min) followed by a basal sample immediately prior to the dosing of test substance (t=0). At t=0, the test substance is dosed intra tracheally into one lung. A special cannula with rounded ending is mounted on a syringe containing the 200 ul air and test substance (1 ml/kg). Via the orifice, the cannula is introduced into the trachea and is forwarded into one of the main bronchi—just passing the bifurcature. During the insertion, the neck is palpated from the exterior to assure intratracheal positioning. The content of the syringe is injected followed by 2 sec pause. Thereafter, the cannula is slowly drawn back. The rats are kept anaesthetized during the test (blood samples for up to 4 hrs) and are euthanized after the experiment.

### IGF-1 Receptor Binding

IGF-1 receptor binding is determined using a by a SPA assay (Scintillation Proximity Assay) microtiterplate antibody capture assay similar to that used for determining the insulin receptor binding of the test compound, with the exception that the IGF1 receptor is used in stead of the insulin receptor, [125I]-human IGF-1 in stead of [125I]-human insulin and an antibody with specificity for the IGF-1 receptor.

1. Single-chain insulin comprising the B- and the A-chain of human insulin or analogues thereof connected by a connecting peptide having from 3-35 amino acid residues, wherein the single-chain insulin comprises at least one PEG group attached to at least one lysine residue in the single-chain insulin molecule and/or to the B-chain N terminal amino acid residue.

2. Single-chain insulin according to claim 1, wherein the connecting peptide has from 5-20 amino acid residues.

3. Single-chain insulin according to claim 1, wherein the connecting peptide has 7-10 amino acid residues.

4. Single-chain insulin according to claim 1 comprising up to 4 PEG groups which may be the same or different.

5. Single-chain insulin according to claim 1 comprising 1-2 PEG groups which may be the same or different.

6. Single-chain insulin according to claim 1 comprising a single PEG group.

7. Single-chain insulin according to claim 1, wherein the PEG group(s) is(are) attached to a lysine residue positioned in one or more of positions B1; B2; B3; B4; B 10; B20; B21; B22; B27; B28; B29; B30; A8; A9; A10; A14; A15; A18; A21; A22; A23 and in the connecting peptide.

8. Single-chain insulin according to claim 1, wherein the PEG group is attached to a lysine residue in position B20, B21 or B22.

9. Single-chain insulin according to claim 1, wherein the PEG group is attached to a lysine residue in position B27; B28; B29; or B30.

10. Single-chain insulin according to claim 1, wherein the PEG group is attached to a lysine residue in position B1; B2; B3; or B4.

11. Single-chain insulin according to claim 1, wherein the PEG group is attached to a lysine residue in position B10.

12. Single-chain insulin according to claim 1, wherein the PEG group is attached to a lysine residue in position A8, A9 or A10.

13. Single-chain insulin according to claim 1, wherein the PEG group is attached to a lysine residue in position A14, A15 or A18.

14. Single-chain insulin according to claim 1, wherein the PEG group is attached to a lysine residue in position A22 or A23.

15. Single-chain insulin according to claim 1, wherein the PEG group is attached to a lysine residue in the connecting peptide.

16. Single-chain insulin according to claim 1, wherein the natural amino acid residue in position A18 is substituted with a Gln residue.

17. Single-chain insulin according to claim 1, wherein the natural amino acid residue in position A21 is substituted with a Gly residue.

18. Single-chain insulin according to claim 1, wherein the natural amino acid residue in position B30 is deleted.

19. Single-chain insulin according to claim 1, wherein the connecting peptide is selected from the group consisting of TGLGSGQ (SEQ ID NO:1); VGLSSGQ (SEQ ID NO:2); VGLSSGK (SEQ ID NO:3); TGLGSGR (SEQ ID NO:4); TGLGKGQ (SEQ ID NO:5); KGLSSGQ (SEQ ID NO:6); VKLSSGQ (SEQ ID NO:7); VGLKSGQ (SEQ NO:8); TGLGKGQ (SEQ ID NO:9) and VGLSKGQ (SEQ ID NO:10).

20. Single-chain insulin according to claim 1, comprising one or more PEG groups comprising a number of (OCH₂CH₂) subunits from 800 to about 1000 subunits.

21. Single-chain insulin according to claim 1, wherein the PEG groups have the formula CH₃—O—(CH₂CH₂O)nCH₂CH₂—O—, where n is an integer from 2 to about 600.

22. Pharmaceutical preparation comprising a biologically active amount of a PEGylated single-chain insulin according to claim 1.

23. Method of reducing the blood glucose level in mammalians by administrating a therapeutically active dose of a pharmaceutical preparation according to claim 22 to a patient in need of such treatment.

24. Method according to claim 23 being a pulmonal administration.

25-26. (canceled)


SEQUENCE LISTING (13 sequences)

SEQ ID NO: 1 - protein, 7 aa, Artificial
  Synthetic sequence
TGLGSGQ

SEQ ID NO: 2 - protein, 7 aa, artificial
  Synthetic sequence
VGLSSGQ

SEQ ID NO: 3 - protein, 7 aa, artificial
  Synthetic sequence
VGLSSGK

SEQ ID NO: 4 - protein, 7 aa, Artificial
  Synthetic sequence
TGLGSGR

SEQ ID NO: 5 - protein, 7 aa, Artificial
  Synthetic sequence
TGLGKGQ

SEQ ID NO: 6 - protein, 7 aa, Artificial
  Synthetic sequence
KGLSSGQ

SEQ ID NO: 7 - protein, 7 aa, Artificial
  Synthetic sequence
VKLSSGQ

SEQ ID NO: 8 - protein, 7 aa, Artificial
  Synthetic sequence
VGLKSGQ

SEQ ID NO: 9 - protein, 7 aa, Artificial
  Synthetic sequence
TGLGKGQ

SEQ ID NO: 10 - protein, 7 aa, Artificial
  Synthetic sequence
VGLSKGQ

SEQ ID NO: 11 - protein, 54 aa, Homo sapiens
  MISC_FEATURE (1)..(1): X = PHE, LYS, ASP OR IS MISSING
XXXXHLCGSXLVEALYLVCXXXGFFYXXXXXGIVEQCCXXICSLYXLEXYCXXX

SEQ ID NO: 12 - protein, 7 aa, Artificial
  Synthetic sequence
XXXXXXX

SEQ ID NO: 13 - protein, 7 aa, Artificial
  Synthetic sequence
XGXGXGX